(19) United States
(12) Patent Application Publication
(10) Pub. No.: US 2025/0186128 A1
(43) Pub. Date: Jun. 12, 2025

(54) Medical Devices for Interventional MRI

(71) Applicants: Cook Medical Technologies LLC, Bloomington, IN (US); Muffin Incorporated dba Cook Advanced Technologies, West Lafayette, IN (US)

(72) Inventors: Ram H. Paul, Jr., Bloomington, IN (US); Sean D. Chambers, Bloomington, IN (US); Neal Fearnot, West Lafayette, IN (US); Joshua F. Krieger, Bloomington, IN (US); Jesse Roll, West Lafayette, IN (US)

(73) Assignees: Cook Medical Technologies LLC, Bloomington, IN (US); Muffin Incorporated dba Cook Advanced Technologies, West Lafayette, IN (US)

(21) Appl. No.: 18/836,215

(22) PCT Filed: Feb. 7, 2023
(86) PCT No.: PCT/US2023/062094

§ 371 Date: Aug. 6, 2024

Related U.S. Application Data
(60) Provisional application No. 63/307,352, filed on Feb. 7, 2022.

(51) Int. Cl. A61B 34/20; A61B 90/00; A61M 25/01; A61M 25/06

(52) U.S. Cl. CPC A61B 34/20; A61B 90/39; A61B 2034/2051; A61B 2090/3954; A61M 25/0127; A61M 2025/0681

(57) ABSTRACT

The disclosure relates to medical devices, methods of performing an interventional medical treatment under MRI, and methods of making a medical device. An example medical device includes an elongate member, a reinforcement member, and a marker. The elongate member has a proximal end, a distal end, and a circumferential wall having an outer surface and an inner surface that defines a lumen. The reinforcement member is disposed within the circumferential wall, extends along a length of the elongate member, is formed of a first material, and has a first susceptibility. The marker is attached to the elongate member, is formed of a second material, and has a second susceptibility that is different from the first susceptibility.

### FIELD

The disclosure relates generally to the field of medical devices. More particularly, the disclosure relates to interventional medical devices useful in performing treatment under magnetic resonance imaging (MRI), methods of performing interventional medical treatment under MRI, and methods of making medical devices. Specific examples described herein relate to introducer sheaths.

### BACKGROUND

Interventional procedures conducted under MRI have several benefits over X-Ray-guided interventions. For example, the patient is not exposed to ionizing radiation. Also, MRI provides the ability to characterize tissue and fluid flow during an interventional procedure. For at least these reasons, the use of interventional MRI is gaining wider acceptance and the number of procedures that can be performed under MRI is generally increasing.

The art provides only a limited number of interventional medical devices suitable for use under MRI, however, which continues to limit growth of the use of interventional MRI procedures. As a result, patients have not yet benefitted fully from interventional MRI technologies and, indeed, are often still limited to less convenient, and potentially less effective, options for certain treatments.

For example, without interventional MRI, addressing some conditions requires the use of multiple imaging modalities over the clinical path from initial testing to treatment. On a practical level, this use of multiple imaging modalities can require multiple patient visits to a healthcare facility. A conventional approach to the treatment of prostate cancer is illustrative—visualization, biopsy, and treatment are performed over the course of three separate patient visits. At a first visit, a scan is completed using a magnetic resonance scanner to produce an image showing the prostate and any abnormalities. The patient then leaves the facility and awaits a review of the image. If abnormalities exist, a second patient visit will occur such that a biopsy sample of the abnormal tissue can be completed. Software is used to merge magnetic resonance images with the procedural ultrasound to provide guidance in conducting the biopsy. This fusion decreases the value of the diagnostic magnetic resonance image. The patient then leaves the facility again and awaits a review of the biopsy sample to determine whether further treatment is required (e.g., if the review results in a positive prostate cancer diagnosis). If further treatment is required, the patient must visit the facility a third time for delivery of the treatment. Completion of these three patient visits often extends over months, prevents the patient from receiving rapid treatment, and increases the overall costs associated with treatment, both to the patient and to the healthcare providers involved. Furthermore, the use of software to merge images from multiple imaging modalities, such as magnetic resonance images and ultrasound images, has drawbacks, such as image overlay or alignment issues and the potential for compression shifting of tissues. Ultimately, these drawbacks of current treatment approaches can limit the overall effectiveness of the treatment. Interventional MRI has the potential to overcome these drawbacks.

A need exists, therefore, for new and improved interventional medical devices useful in performing treatment under MRI, methods of performing interventional medical treatment under MRI, and methods of making medical devices.

### BRIEF SUMMARY OF SELECTED EXAMPLES

Various example interventional medical devices useful in performing treatment under MRI, methods of performing interventional medical treatment under MRI, and methods of making medical devices are described herein.

An example medical device comprises an elongate member having a proximal end, a distal end, and a circumferential wall having an outer surface and an inner surface that defines a lumen; a reinforcement member disposed within the circumferential wall, extending along a length of the elongate member and formed of a first material and having a first susceptibility; and a marker attached to the elongate member and formed of a second material and having a second susceptibility that is different from the first susceptibility.

Another example medical device comprises an elongate member having a proximal end, a distal end, and a circumferential wall having an outer surface and an inner surface that defines a lumen; a reinforcement member disposed within the circumferential wall, extending along a length of the elongate member, and formed of a first material having a first susceptibility; and a marker disposed within the circumferential wall distal to the reinforcement member and formed of a second material different from the first material and having a second susceptibility that is greater than the first susceptibility.

Another example medical device comprises an elongate member having a proximal end, a distal end, and a circumferential wall having an outer surface and an inner surface that defines a lumen; a reinforcement member comprising a coil disposed within the circumferential wall, extending along a length of the elongate member, and formed of a first material having a first susceptibility; and a marker disposed within the circumferential wall distal to the reinforcement member and formed of a second material different from the first material and having a second susceptibility that is greater than the first susceptibility.

Another example medical device comprises an elongate member having a proximal end, a distal end, and a circumferential wall having an outer surface and an inner surface that defines a lumen; a reinforcement member disposed within the circumferential wall, extending along a length of the elongate member and formed of a first material having a first magnetic susceptibility; and a marker attached to the elongate member and formed of a second material having a second magnetic susceptibility that is greater than the first magnetic susceptibility.

Another example medical device comprises an elongate member having a proximal end, a distal end, and a circumferential wall having an outer surface and an inner surface that defines a lumen; a reinforcement member disposed within the circumferential wall, extending along a length of the elongate member, and formed of a first material having a first magnetic susceptibility; and a marker disposed within the circumferential wall distal to the reinforcement member and formed of a second material different from the first material and having a second magnetic susceptibility that is greater than the first magnetic susceptibility.

Another example medical device comprises an elongate member having a proximal end, a distal end, and a circumferential wall having an outer surface and an inner surface that defines a lumen; a reinforcement member comprising a coil disposed within the circumferential wall, extending along a length of the elongate member, and formed of a first material having a first magnetic susceptibility; and a marker disposed within the circumferential wall distal to the reinforcement member and formed of a second material different from the first material and having a second magnetic susceptibility that is greater than the first magnetic susceptibility.

Another example medical device comprises an elongate member having a proximal end, a distal end, and a circumferential wall having an outer surface and an inner surface that defines a lumen; a reinforcement member comprising a mesh disposed within the circumferential wall, extending along a length of the elongate member, and formed of a first material having a first magnetic susceptibility; and a marker disposed within the circumferential wall distal to the reinforcement member and formed of a second material different from the first material and having a second magnetic susceptibility that is greater than the first magnetic susceptibility.

Various example methods of performing an interventional medical treatment under MRI are also included.

Various example methods of making a medical device are described.

Additional understanding of these and other example interventional medical devices, methods of performing interventional medical treatment under MRI, and methods of making a medical device can be obtained by review of the detailed description of selected examples, below, and the references drawings.

### DESCRIPTION OF FIGURES

FIG. 1 is a side view of an example medical device.

FIG. 2 is a magnified sectional view of the medical device illustrated in FIG. 1.

FIG. 2A is a magnified sectional view of an alternative medical device.

FIG. 3 is a magnified sectional view of the distal end of the medical device illustrated in FIG. 1.

FIG. 4 is a sectional view of the tubular medical device illustrated in FIG. 1, taken along line A-A.

FIG. 5 is a sectional view of the tubular medical device illustrated in FIG. 1, taken along line B-B.

FIG. 6 is a sectional view of the tubular medical device illustrated in FIG. 1, taken along line C-C.

FIG. 7 is a sectional view of the tubular medical device illustrated in FIG. 1, taken along line D-D.

FIG. 8 is a side view of an example introducer sheath.

FIG. 9 is a side view of another example medical device, including a magnified view of the distal portion of the medical device.

FIG. 10 is a magnetic resonance image of a prior art medical device and a medical device according to an embodiment.

FIG. 11 is a magnetic resonance image of a prior art medical device and a medical device according to an embodiment.

FIG. 12 is a magnetic resonance image of a prior art medical device and a medical device according to an embodiment.

FIG. 13 is a magnetic resonance image of a prior art medical device and a medical device according to an embodiment.

FIG. 14 is a magnetic resonance image of three medical devices according to embodiments.

FIG. 15 is a flowchart illustration of an example method of performing an interventional medical treatment.

### DETAILED DESCRIPTION OF SELECTED EXAMPLES

The following detailed description and the appended drawings describe and illustrate various example interventional medical devices, imaging methods, methods of performing interventional medical treatment under MRI, and methods of making medical devices. The description and illustration of these examples are provided to enable one skilled in the art to make and use an interventional medical device and to perform imaging methods, methods of performing interventional medical treatment under MRI, and methods of making interventional medical devices. They are not intended to limit the scope of the invention, or its protection, in any manner. The invention is capable of being practiced or carried out in various ways and the examples described and illustrated herein are not considered exhaustive.

As used herein, the term “attached” refers to one member being secured to another member such that the members do not completely separate from each other during use performed in accordance with the intended use of an item that includes the members in their attached form.

As used herein, the term “circumference” refers to an external enclosing boundary of a body, element, or feature and does not impart any structural configuration on the body, element, or feature.

As used herein, the term “magnetic susceptibility” refers to the intrinsic property of a material that relates to how much the material will become magnetized in an applied magnetic field.

As used herein, the term “marker” refers to a discrete deposit of a first material on a second material such that the first material is visible under MRI and is distinguishable from the second material under MRI, a portion of an interventional device in which a first material has been incorporated into a second material such that the combination of the first and second materials is visible under MRI and is distinguishable from the second material under MRI, and a portion of an interventional device in which a material that forms a portion of an interventional device has been manipulated such that the portion is visible under MRI and is distinguishable from the remainder of the interventional device under MRI.

As used herein, the term “passive,” in relation to a marker, refers to a marker that is either unpowered or powered exclusively by the electromagnetic field of a magnetic resonance scanner.

As used herein, the term “susceptibility,” when not immediately preceded by “magnetic,” refers to the ability of an element to influence an external magnetic field. Susceptibility is dependent on various properties of an element, including the size, density, geometric configuration, volume, and other physical properties of the element, and the magnetic susceptibility of the material of which the element is formed.

As used herein, the term “treatment” refers to a medical procedure performed on or in a portion of a body of a patient. Examples of treatments include delivery of an agent to a site within a body vessel, modification of a local environment inside of a body vessel such as by heating or cooling, and removal of a tissue or portion of a tissue from a site within a body of a patient (i.e., biopsy).

As used herein, the term “wire” refers to a strand or rod of material. The term does not require any particular cross-sectional shape, composition, physical properties, or production method by which a referenced element was made.

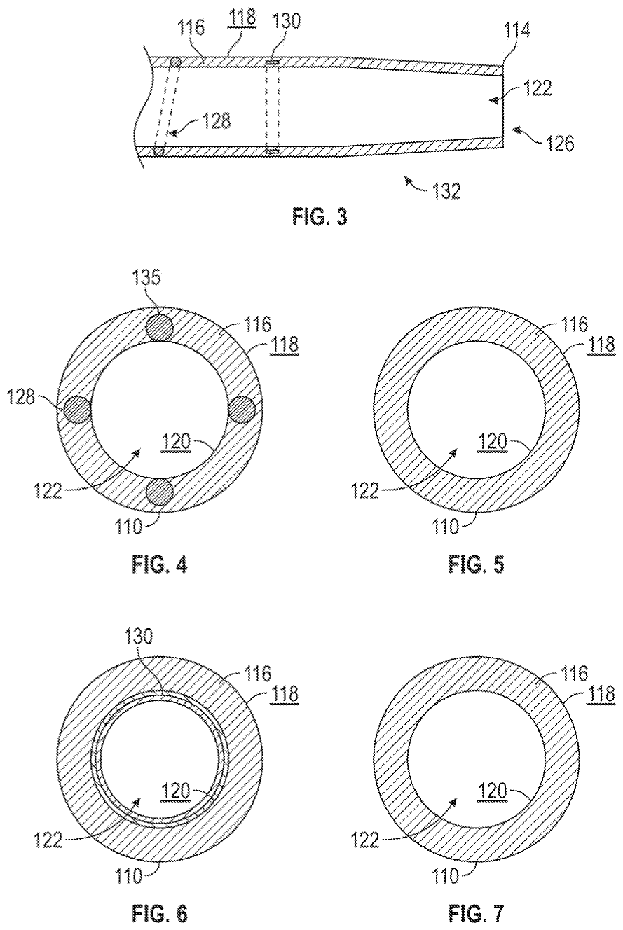

FIGS. 1, 2, 3, 4, 5, 6, and 7 illustrate an example medical device 100. The medical device 100 includes an elongate member 110 having a proximal end 112, a distal end 114, a lengthwise axis 102 extending between the proximal end 112 and the distal end 114, and a length 104 that extends from the proximal end 112 to the distal end 114. The elongate member 110 has a circumferential wall 116 having an outer surface 118 and an inner surface 120 that defines a lumen 122. The lumen 122 extends from a proximal opening 124 on the proximal end 112 to a distal opening 126 on the distal end 114. A reinforcement member 128 is disposed within the circumferential wall 116 and extends along a length 106 of the elongate member 110. The reinforcement member 128 is formed of a first material having a first susceptibility. A marker 130 is attached to the elongate member 110 and is formed of a second material having a second susceptibility that is different from the first susceptibility. The first and second materials can be the same or different. Thus, the first and second materials can have the same or different magnetic susceptibility.

The elongate member 110 is formed of a polymeric material such that the reinforcement member 128 can be disposed within the circumferential wall 116 during fabrication. Any polymeric material can be used and a skilled artisan will be able to select a suitable polymeric material for the elongate member in a medical device according to a particular embodiment based on various considerations, including desired any desired handling and performance characteristics of the medical device, such as torqueability and pushability. Examples of suitable polymeric materials include, but are not limited to, heat-formable polymeric materials, such as polyamide materials. These polymeric materials are considered desirable at least because of their ability to melt and flow between and around elements during a heat forming or heat shrinking process. Nylon is considered particularly advantageous at least because of its ready availability and well-characterized nature.

The elongate member 110 can have any suitable form and a skilled artisan will be able to select a suitable form for a medical device according to a particular embodiment based on several considerations, including the intended use of the medical device and the nature of any body vessel within which the medical device is intended to be placed. In the illustrated example, the elongate member 110 includes a distal portion 132 that defines a taper 134 along the outer surface 118 such that the distal opening 126 has the same diameter as the diameter of the proximal opening 124. For example, the thickness of circumferential wall 116 can gradually become smaller over the distal portion 132 while the inner diameter of the lumen 122 is continuous over the distal portion 132. Alternatively, the elongate member 110 can include a distal portion 132 that defines a taper 134 along both the outer surface 118 and the inner surface 120 such that the distal opening 126 has a smaller diameter than the diameter of the proximal opening 124. Also alternatively, the elongate member 110 can have substantially continuous outer and inner diameters along its length.

The elongate member can have any suitable axial length and a skilled artisan will be able to select a suitable length for a medical device according to a particular embodiment based on various considerations, including the intended use of the medical device and the nature of any body vessel within which the medical device is intended to be placed. Examples of lengths considered suitable for a elongate member in a medical device according to the invention include, but are not limited to, lengths equal to, greater than, less than, or about 100 centimeters, 110 centimeters, 120 centimeters, 130 centimeters, 140 centimeters, 240 centimeters, 250 centimeters, 260 centimeters, 270 centimeters, 280 centimeters, between about 50 centimeters and about 350 centimeters, between about 100 centimeters and about 280 centimeters, between about 120 centimeters and about 260 centimeters, and any other length considered suitable for a medical device according to a particular embodiment.

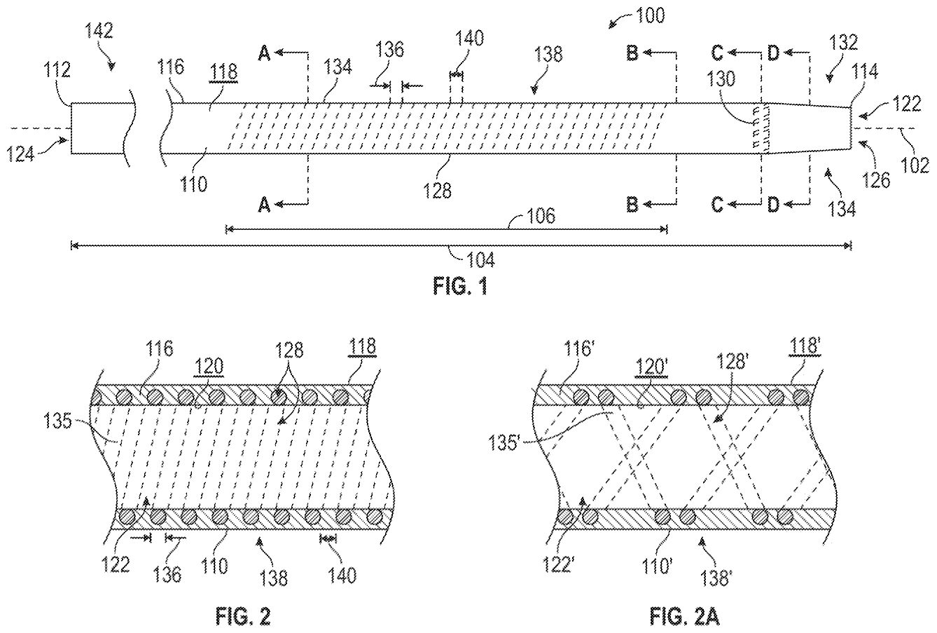

As best illustrated in FIGS. 2 and 4, reinforcement member 128 is embedded within the thickness of the circumferential wall 116 of the elongate member 110 such that the reinforcement member 128 is disposed entirely within the thickness of the elongate member 110 and such that no portion of the reinforcement member 128 breaches any portion of either the outer surface 118 or the inner surface 120 of the elongate member 110. The reinforcement member 128 extends around the lumen 122 and along an axial length 106 of the elongate member 110. The reinforcement member 128 is formed of a metal or an alloy and has a magnetic susceptibility that is less than the magnetic susceptibility of the marker 130.

The reinforcement member 128 can have any suitable structural configuration relative to the elongate member 110 that provides the desired extension around the lumen 122 and along an axial length 106 of the elongate member 110. A skilled artisan will be able to select a desirable structural configuration for the reinforcement member in a medical device according to a particular embodiment based on various considerations, including any desired handling characteristics of the medical device. In the illustrated embodiment, the reinforcement member 110 comprises a wire 135 having a thickness 136 and formed into a coil 138 extending around the lumen 122 and along an axial length 106 of the elongate member 110. Adjacent turns of the coil 138 are separated by a gap 140. The reinforcement member in a medical device according to a particular embodiment can have other another structural configuration, though. For example, the reinforcement member can form an interrupted coil, a coil having a variable pitch along its axial length, a coil having a variable diameter along its axial length, and combinations of these structural configurations. FIG. 2A illustrates an alternative elongate member 110′ in which the reinforcement member 128′ comprises a mesh 138′ of wire members 135′. As with the embodiment illustrated in FIG. 2, the reinforcement member 128′ is embedded within the thickness of the circumferential wall 116′ such that the reinforcement member 128′ is disposed entirely within the thickness of the elongate member 110′, leaving no portion of the mesh 138′ that breaches the outer surface 118′ or the inner surface 120′ of the elongate member 110′. Also similar to the embodiment illustrated in FIG. 2, the reinforcement member 128′ extends around the lumen 122′ of the elongate member 110′. Other examples of suitable structural configurations for the reinforcement member include, but are not limited to, a secondary structure that forms multiple cells, such as hexagon-shaped or octagon-shaped cells, or those in which a reinforcement member comprises a braided material that extends around a lumen of an elongate member (e.g., partially, entirely) and is embedded (e.g., entirely) within a thickness of a circumferential wall of the elongate member.

The reinforcement member 128 has a susceptibility that is different from the susceptibility of the marker 130. Accordingly, the reinforcement member 128 can be formed of any metal, alloy, or other material that provides the desired relative susceptibility as compared to the susceptibility of the marker 130. A skilled artisan will be able to select a suitable material for the reinforcement member in a medical device according to a particular embodiment based on various considerations, including the composition of the marker in the medical device. Suitable pairings of materials, properties, and structural configurations for the reinforcement member and the marker in medical devices according to the invention are described in detail below. Examples of suitable materials for the reinforcement member include, but are not limited to, Titanium, alloys, such as alloys containing less than or equal to 1% Iron by weight, such as Cobalt Chromium alloys, polymeric materials, such as polyether ether ketone (PEEK), and other materials.

The reinforcement member 128 can extend along any axial length of the elongate member 110 and a skilled artisan will be able to select a suitable axial length for a medical device according to a particular embodiment based on various considerations, including any desired handling characteristics of the medical device. As best illustrated in FIG. 1, the reinforcement member 128 in the example medical device 100 extends along an axial length 106 that is less than the total axial length 104 of the elongate member 110. A proximal portion 142 of the elongate member 110 includes the proximal end 112 and is free of the reinforcement member 128. That is, the reinforcement member 128 terminates and does not extend into the proximal portion 142 of the elongate member 110. Similarly, a distal portion 132 of the elongate member 110 includes the distal end 114 and is free of the reinforcement member 128. Thus, the reinforcement member 128 terminates and does not extend into the distal portion 132 of the elongate member 110. It is considered advantageous to axially separate the marker 130 from the distal end of the reinforcement member 128. This axial separation of these elements provides desirable imaging properties in light of the relative susceptibilities of the materials that form these elements, and also provides a desirable stiffness to the distal end relative to the portion of the elongate member that includes the reinforcement member without sacrificing the advantageous handling characteristics imparted onto other portions of the elongate member that include the reinforcement member. Thus, as best illustrated in FIG. 1, distal portion 132 of the elongate member 110 is free of the reinforcement member 128. Alternatively, however, a marker can be formed as a portion of a reinforcement member (e.g., attached to, formed from the same material) such that the material that forms the marker, or the portion of the material forming the marker, has a susceptibility that is different from the susceptibility of the material that forms the remainder of the reinforcement member, as described herein.

Examples of suitable axial lengths for a reinforcement member in a medical device according to a particular embodiment include, but are not limited to, 100% of the axial length of the elongate member of the medical device, about 100% of the axial length of the elongate member of the medical device, less than 100% of the axial length of the elongate member of the medical device, about 95% of the axial length of the elongate member of the medical device, about 90% of the axial length of the elongate member of the medical device, about 85% of the axial length of the elongate member of the medical device, and about 80% of the axial length of the elongate member of the medical device. Other examples of suitable axial lengths for a reinforcement member in a medical device according to a particular embodiment include, but are not limited to, between about 50% and about 100% of the axial length of the elongate member of the medical device, between about 60% and about 95% of the axial length of the elongate member of the medical device, between about 70% and about 95% of the axial length of the elongate member of the medical device, between about 80% and about 95% of the axial length of the elongate member of the medical device, and between about 90% and about 95% of the axial length of the elongate member of the medical device.

As best illustrated in FIGS. 1 and 6, marker 130 is embedded within the thickness of the circumferential wall 116 of the elongate member 110 such that the marker 130 is disposed entirely within the thickness of the elongate member 110 and such that no portion of the marker 130 breaches any portion of either the outer surface 118 or the inner surface 120 of the elongate member 110. In the illustrated embodiment, the marker 130 extends around the lumen 122 of the elongate member 110. Marker 130 is a passive marker and is formed of a metal or an alloy and has a susceptibility that is different from the susceptibility of the reinforcement member 128.

Marker 130 can have any structural configuration, and a skilled artisan will be able to select a suitable structural configuration for a medical device according to a particular embodiment based on various considerations, including any desired visualization characteristics when the medical device is used with imaging modalities, such as MRI. Examples of suitable configurations include, but are not limited to, a ring, a strip, a plug, a twisted band, a twisted ring, multiple bands attached to each other, multiple rings attached to each other, and other configurations. A circumferential band of material, as illustrated in FIGS. 1 and 6, is considered particularly advantageous. A medical device can include any number of markers, too, and a skilled artisan will be able to select a suitable number of markers for a medical device according to a particular embodiment based on various considerations, including any desired visualization patterns when the medical device is used with imaging modalities, such as MRI. Examples of suitable numbers include, but are not limited to, one, more than 1, two, a plurality, three, more than three, four, five, six, seven, eight, nine, ten, and more than ten.

The marker 130 can be disposed at any suitable position relative to reinforcement member 128, and a skilled artisan will be able to select a suitable position for a marker relative to the reinforcement member in a medical device according to a particular embodiment based on various considerations, including any desired visualization patterns when the medical device is used with imaging modalities, such as MRI. As best illustrated in FIG. 1, the marker 130 in the example medical device 100 is positioned distal to the reinforcement member 128, in the distal portion 132 of the elongate member 110. Other examples of suitable positions include, but are not limited to, within an axial portion of the elongate member that includes the reinforcement member such that the marker overlaps the reinforcement member, within an axial portion of the elongate member that is proximal to the reinforcement member, at the distal end of the elongate member, at the proximal end of the elongate member, and combinations of these positions with multiple markers.

The marker 130 has a susceptibility that is different from the magnetic susceptibility of the reinforcement member 128. Accordingly, the marker can be formed of any metal, alloy, or other material that provides the desired relative susceptibility as compared to the susceptibility of the reinforcement member 128. A skilled artisan will be able to select a suitable material for the marker in a medical device according to a particular embodiment based on various considerations, including the composition of the reinforcement member in the medical device. Suitable pairings of materials for the reinforcement member and the marker in medical devices according to the invention are described in detail below. Examples of suitable materials for the marker include, but are not limited to, metals, such as Titanium, Nickel, and other metals, alloys, such as stainless steel alloys, including 304V stainless steel and 316LVM stainless steel, ferromagnetic materials, paramagnetic materials, alloys containing at least 50% Iron by weight, ferromagnetic and paramagnetic compounds, such as those in powder form, Tantalum powder, Barium Sulfate, Bismuth Oxychloride, Tungsten, Iron Oxide nanoparticles, functionalized magnetite, Gadolinium, Ferritic Stainless Steel, Ferritic Stainless Steel powders, 316 Stainless Steel, nylon compounded with another material, such as tungsten, bismuth, and others, and any other material considered suitable for a particular embodiment. Alternative to incorporating a marker into the circumferential wall of an elongate member of a medical device, alternative embodiments can include a marker disposed on a surface of the elongate member, such as an inner or outer surface. For example, a marker can be printed onto or adhered to an inner or outer surface of an elongate member of a medical device. For example, an ink containing a material having a magnetic susceptibility that is greater than the magnetic susceptibility of the reinforcement member in a medical device, such as an ink containing magnetic particles, an ink containing Iron Oxide nanoparticles, or an ink containing Iron Oxide nanoparticles bound to phospholipids, can be printed onto an outer surface and/or an inner surface of an elongate member to form a marker in a medical device according to an embodiment. A marker can be disposed on a surface by other suitable processes, too, such as chemical vapor deposition. Also alternatively, a tape including a material having a magnetic susceptibility that is greater than the magnetic susceptibility of the reinforcement member in a medical device, such as magnetic tape, can be adhered to an outer surface or an inner surface of an elongate member to form a marker in a medical device according to an embodiment. Selection of a marker, or markers, to include in a medical device according to a particular embodiment can also be based upon the field strength, or field strengths, within which the medical device is intended to be used. For example, a medical device that includes a marker can be utilized to complete one, or more than one, interventional procedure under MRI utilizing one or more field strengths (0.55 T, 1.5 T, or 3.0 T). Material or materials can be selected for a marker or markers in a medical device according to an embodiment based on these expected field strengths and the expected visual artifacts produced by a marker or markers formed of a particular material and having a particular structural configuration.

The reinforcement member in medical devices according to the invention has a susceptibility that is different from the susceptibility of a marker in the medical device. Thus, the marker in medical devices according to the invention has a susceptibility that is different from the susceptibility of the reinforcement in the medical device. Any pairing of materials for these elements that provides this relative relationship of the susceptibilities for these elements, which is considered critical to the performance of medical devices according to the invention, can be used in a medical device according to a particular embodiment. Indeed, the reinforcement member and the marker can be formed of the same or different materials as long as the relative relationship of the susceptibilities for these elements is provided. In some embodiments, different materials having different magnetic susceptibilities are used for the reinforcement member and the marker. In these embodiments, the reinforcement member and the marker have different susceptibilities and are formed of materials having different magnetic susceptibilities. For these embodiments, a skilled artisan will be able to select a material for one of these elements in a medical device according to a particular embodiment based on various considerations, including the composition of the other of these elements and any desired performance characteristics or imaging characteristics for the medical device. Examples of suitable pairings of different materials for the reinforcement member and the marker include, but are not limited to, a first material for the reinforcement member and a second, different material for the marker, such as a paramagnetic material for the reinforcement member and a ferromagnetic material for the marker, an alloy containing less than or equal to 1% Iron by weight for the reinforcement member and an alloy containing at least 50% Iron by weight for the marker, a Cobalt Chromium alloy for the reinforcement member and a stainless steel for the marker, and a Nickel Cobalt alloy, such as MP35N, for the reinforcement member and a stainless steel, such as 304V stainless steel of 316LVM stainless steel, for the marker. In other embodiments, the reinforcement member and the marker are formed of the same material. In these embodiments, while the reinforcement member and the marker have different susceptibilities, the reinforcement member and the marker have the same magnetic susceptibility. For example, in the illustrated embodiment, the reinforcement member 128 and the marker 130 can be formed of the same material, giving the reinforcement member 128 and the marker 130 the same magnetic susceptibility. To provide the different susceptibilities for these elements 128, 130, one can be work-hardened or manipulated in some manner that provides a susceptibility that is different from the susceptibility of the other.

Examples of suitable materials for the reinforcement member include, but are not limited to, metals, such as Titanium, alloys, such as stainless steel, Nickel-containing alloys, Cobalt-containing alloys, alloys containing less than or equal to 1% Iron by weight, such as Cobalt Chromium alloys, polymeric materials, such as polyether ether ketone (PEEK), glass fibers, and the like.

The medical device 100 can include additional optional components. For example, a liner, such as a liner formed of a lubricious fluoropolymer, such as polytetrafluoroethylene (PTFE), can be disposed on the inner surface 120 of the circumferential wall. Also, a handle, connector, or other component can be attached to the proximal end 112 of the elongate member 110 to aid in handling of the medical device 100 during use or to facilitate use of the medical device 100 with other medical devices, such as catheters and the like.

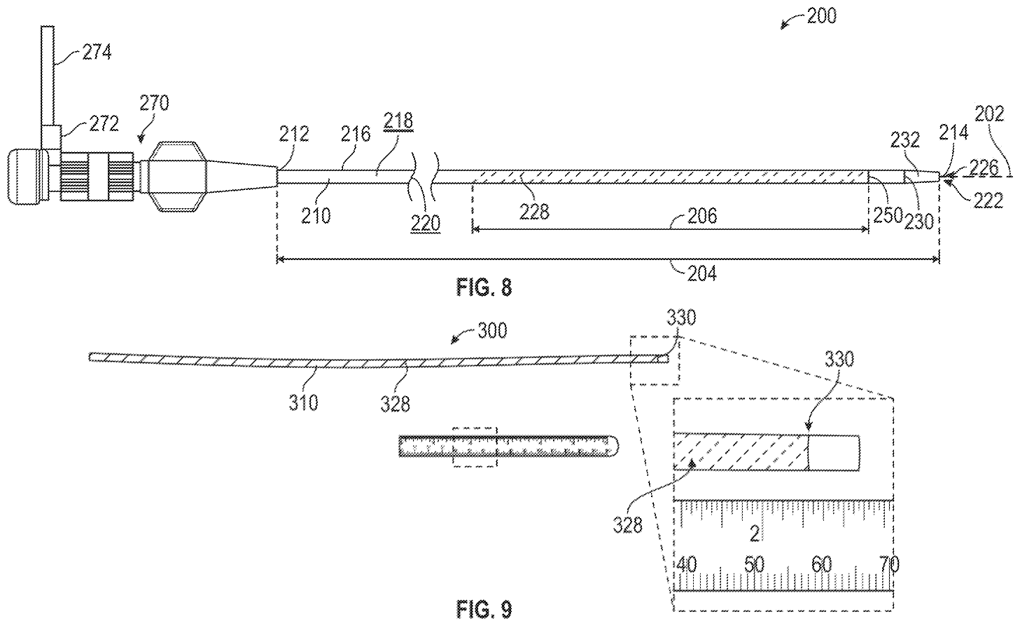

Medical devices according to the invention can take various configurations, depending on the intended use of the particular medical device. The medical device illustrated in FIGS. 1, 2, 3, 4, 5, 6, and 7 is one example configuration. FIG. 8 illustrates another example medical device 200. The medical device 200 is an introducer sheath useful in the placement, delivery, or deployment of another interventional medical device, such as a catheter, a stent, a stent graft, a valve, a filter, a coil, an embolization device, such as a bead or beads or a particle or particles, or the like, in a body vessel of an animal, such as a human. The medical device 200 is similar to the medical device 100 described above, except as detailed below. Thus, medical device 200 includes an elongate member 210 having a proximal end 212, a distal end 214, a lengthwise axis 202 extending between the proximal end 212 and the distal end 214, and a length 204 that extends from the proximal end 212 to the distal end 214. The elongate member 210 has a circumferential wall 216 having an outer surface 218 and an inner surface 220 that defines a lumen 222. The lumen 222 extends from a proximal opening (not illustrated in the Figure) on the proximal end 212 to a distal opening 226 on the distal end 214. A reinforcement member 228 is disposed within the circumferential wall 216 and extends along a length 206 of the elongate member 210. The reinforcement member 228 is formed of a first material having a first susceptibility. A first marker 230 is attached to the elongate member 210 and is formed of a second material having a second susceptibility. A second marker 250 is attached to the elongate member 210 and is formed of a third material having a third susceptibility. The first, second, and third materials can be the same or different. The second and third susceptibilities can be the same or different, but each is different from the first susceptibility. A dilator having a tapered distal end can be disposed longitudinally through the lumen 222 of the elongate member 210 for accessing and dilating a vascular access site, e.g., over a conventional wire guide (not shown).

In this example, a connector hub 270 is attached about the proximal end 212 of the elongate member 210. Connector hub 270 may include a conventional silicone disk (not illustrated in the Figure) for preventing backflow of fluids through the connector hub 270 during use of the medical device 200. Connector hub 270 also includes a side arm 272, to which a polymeric tube 274 and other components, such as a Luer lock connector, may be connected for introducing and aspirating fluids therethrough in conventional fashion.

In this example, medical device 200 includes a first marker 230 and a second marker 250. Each of the first marker 230 and the second marker 250 is disposed distal to the reinforcement member 228. This positioning of multiple markers, each of which has a susceptibility that is different from the susceptibility of the reinforcement member 228, is considered advantageous at least because it positions the markers in a location on the axial length of the medical device 200 that is ultimately positioned at or near a point of treatment in a body vessel during use of the medical device 200 and distal to the reinforcement member. When used with imaging modalities, such as MRI, this positioning, along with the relative susceptibilities, provides desirable imaging artifacts than can be used for confirmation of placement of the distal portion 232 of the medical device 200.

### EXAMPLES

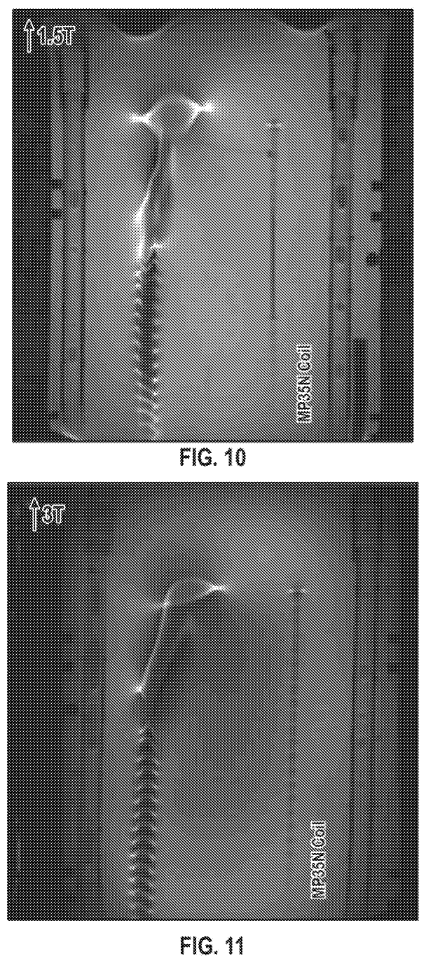

FIG. 9 illustrates an example medical device 300 that includes an elongate member 310, a reinforcement member 328 comprising a coil formed of MP35N Nickel Cobalt, and a marker 330 disposed distal to the reinforcement member 328 and comprising a circumferential ring of 304V stainless steel.

Each of FIG. 10 and FIG. 11 illustrates artifacts in a magnetic resonance image from a prior art medical device having a reinforcement member comprising a stainless steel coil (left side in both Figures) and the medical device 300 illustrated in FIG. 9 (right side in both Figures). FIG. 10 is an image taken under a spin echo imaging sequence at 1.5 T in copper sulfate as a phantom solution. FIG. 11 is an image taken under a spin echo imaging sequence at 3 T in copper sulfate as a phantom solution.

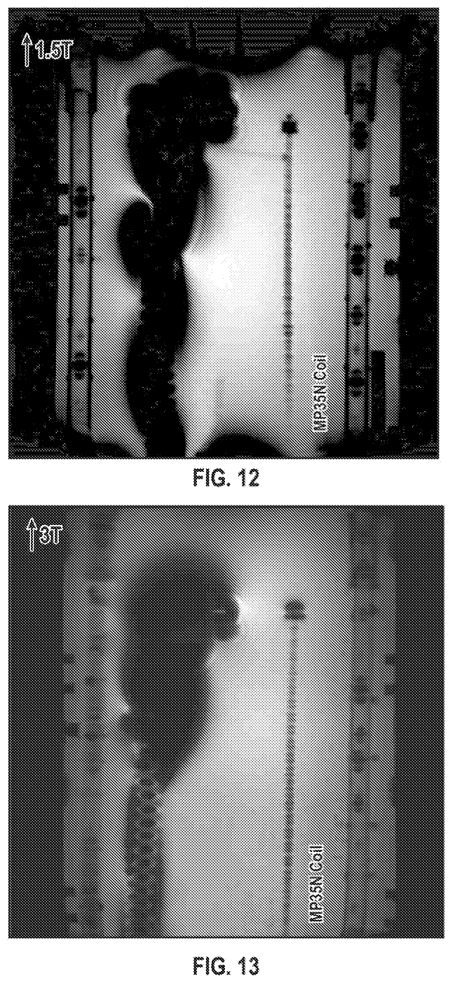

Each of FIG. 12 and FIG. 13 illustrates artifacts in a magnetic resonance image from a prior art medical device having a reinforcement member comprising a stainless steel coil (left side in both Figures) and the medical device 300 illustrated in FIG. 9 (right side in both Figures). FIG. 12 is an image taken under a gradient refocusing echo (GRE) imaging sequence at 1.5 T in copper sulfate as a phantom solution. FIG. 13 is an image taken under a gradient refocusing echo (GRE) imaging sequence at 3 T in copper sulfate as a phantom solution.

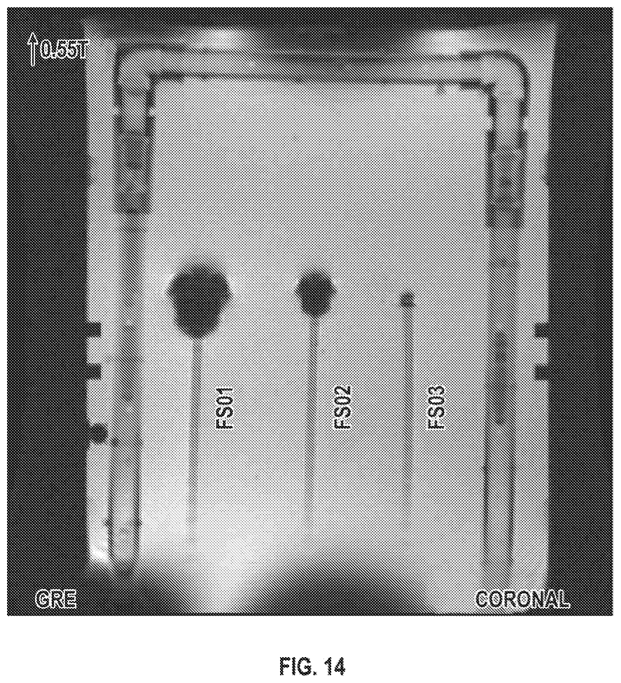

FIG. 14 illustrates artifacts in a magnetic resonance image from first, second, and third example medical devices according to embodiments of the invention. The first example medical device comprises a coil formed of MP35N Nickel Cobalt and a single marker disposed distal to the reinforcement member and comprising a circumferential band of 304V stainless steel. The second example medical device comprises a coil formed of MP35N Nickel Cobalt and a single marker disposed distal to the reinforcement member and comprising a twisted circumferential band of 304V stainless steel. The third example medical device comprises a coil formed of MP35N Nickel Cobalt and a single marker disposed distal to the reinforcement member and comprising a circumferential band of 316LVM stainless steel.

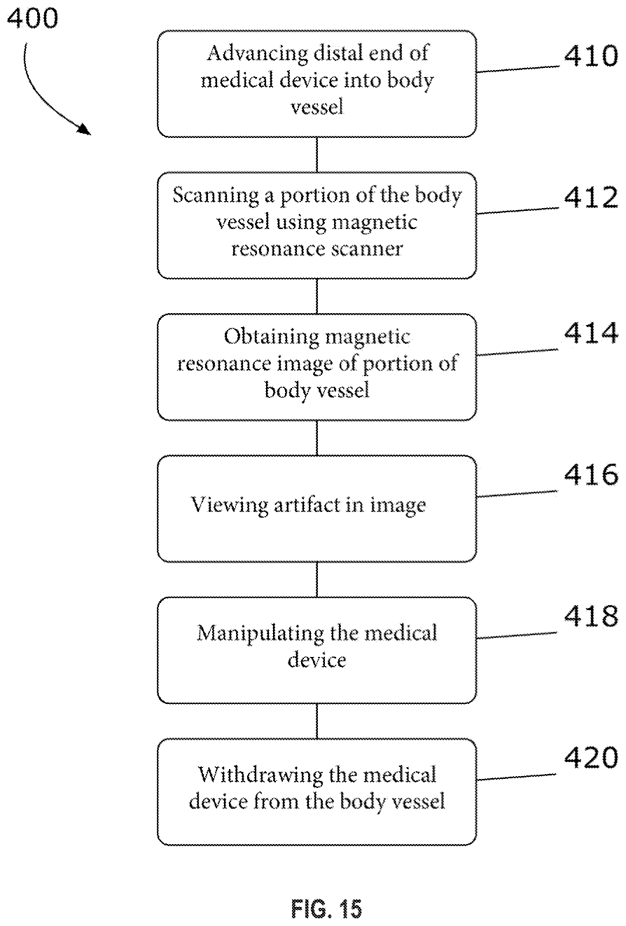

FIG. 15 is a schematic illustration of an example method 400 of performing an interventional medical treatment under MRI.

An initial step 410 comprises advancing the distal end of a medical device to a first location within a body vessel of an animal, such as a human and until a marker of the medical device is disposed at a second location within the body vessel. Another step 412 comprises scanning a portion of the body vessel that includes the second location within the body vessel using a magnetic resonance scanner. Another step 414 comprises obtaining a magnetic resonance image of the portion of the body vessel such that the image includes an artifact indicative of the presence of the marker within the portion of the body vessel. Another step 416 comprises viewing the artifact in the image generated by the presence of the marker. Another step 418 comprises manipulating the medical device based on the location of the artifact relative to the body vessel. Another step 420 comprises withdrawing the medical device from the body vessel.

The step 410 of advancing the distal end of a medical device is performed using a medical device according to an embodiment of the invention, such as any of the example medical devices described herein. Thus, the medical device includes an elongate member defining a lumen, a reinforcement member disposed within a circumferential wall of the elongate member and comprising a metal or alloy having a first magnetic susceptibility, and at least one marker disposed within or otherwise attached to the circumferential wall of the elongate member and comprising a metal or alloy having a second magnetic susceptibility that is greater than the first magnetic susceptibility.

Step 412 can be accomplished by scanning the portion of the body vessel using a magnetic resonance scanner having any suitable number and type of magnetic resonance image parameters, such as gradient refocusing echo imaging, spin echo imaging, true fast imaging with steady-state precession, fast low flip angle shot spoiled gradient-echo imaging, field strengths, such as 0.55 T, 1.5 T, 3 T, between about 0.055 T and 1.5 T, and fields less than 1 T, slice thickness, flip angle, field-of-view, resolution, gradient fields, and any other image parameter considered suitable for a particular embodiment.

Step 414 can be accomplished by obtaining the magnetic resonance image from the magnetic resonance scanner used in step 412.

Step 416 can be accomplished by reviewing the magnetic resonance image obtained in step 414 and identifying an artifact in the image based on the presence of the marker in the medical device.

Step 418 is performed in a manner that achieves, or that contributes to the achievement of, a desired clinical outcome of the method 400 of performing an interventional medical treatment. As such, the nature of the step 418 of manipulating the medical device will depend on the nature of the medical device and the desired clinical outcome. Examples of suitable actions that can be performed for this step include, but are not limited to, axially advancing the medical device within the bodily passage, rotating the medical device within the bodily passage, advancing another medical device through the lumen of the elongate member of the medical device, deploying another medical device from a position within the lumen of the elongate member of the medical device, and axially withdrawing a portion of the medical device to allow another medical device or a portion of another medical device to radially expand within the body vessel. In alternative embodiments, step 418 can be omitted from method 400 when manipulation of the interventional medical device is not desired.

The step 420 of withdrawing the medical device from the body vessel is performed by axially retracting the medical device from the body vessel until the distal end of the medical device is no longer disposed within the body vessel.

Any of the steps being performed by a magnetic resonance scanning can be accomplished using any suitable magnetic resonance scanner, such as conventional magnetic resonance scanners, magnetic resonance scanners that utilize 0.55 T fields, 1.5 T fields, 3 T fields, fields between about 0.055 T and 1.5 T, fields less than 1 T, and any other magnetic resonance scanner considered suitable for a particular embodiment.

A method of making a medical device includes disposing a reinforcement member formed of a first material and having a first susceptibility within a circumferential wall of an elongate member and attaching a marker formed of a second material and having a second susceptibility that is different from the first susceptibility to the circumferential wall of the elongate member. The first material and the second material can be the same or different. Thus, the first material and the second material can have the same or different magnetic susceptibilities. In one example method, the first and second materials are the same and an additional step of work hardening the marker is included to provide the different susceptibilities for the marker and the reinforcement member.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A medical device, comprising:
an elongate member having a proximal end, a distal end, and a circumferential wall having an outer surface and an inner surface that defines a lumen;
a reinforcement member disposed within the circumferential wall, extending along a length of the elongate member, and formed of a first material and having a first susceptibility; and
a marker attached to the elongate member and formed of a second material and having a second susceptibility that is different from the first susceptibility.

2. The medical device of claim 1, wherein the first material and the second material are the same.

3. The medical device of claim 1, wherein the first material and the second material are different.

4. The medical device of claim 3, wherein the first material comprises a paramagnetic material and the second material comprises a ferromagnetic material.

5. The medical device of claim 3, wherein the first material comprises an alloy containing less than or equal to 1% Iron by weight and the second material comprises an alloy containing at least 50% Iron by weight.

6. The medical device of claim 3, wherein the first material comprises a Cobalt Chromium alloy and the second material comprises a stainless steel alloy.

7. The medical device of claim 3, wherein the first material comprises a Nickel Cobalt alloy and the second material comprises a stainless steel alloy.

8. The medical device of claim 7, wherein the second material comprises one of 304V stainless steel and 316LVM stainless steel.

9. The medical device of claim 1, wherein the reinforcement member comprises a coil.

10. The medical device of claim 1, wherein the reinforcement member comprises a mesh.

11. The medical device of claim 1, wherein the marker comprises a band.

12. The medical device of claim 1, wherein the marker comprises a twisted ring.

13. The medical device of claim 1, wherein the marker is disposed within the circumferential wall.

14. The medical device of claim 1, wherein the marker is disposed distal to the reinforcement member.

15. The medical device of claim 1, wherein the marker comprises a first marker; and
further comprising a second marker formed of a third material and having a third susceptibility that is different from the first susceptibility.

16. The medical device of claim 15, wherein the first marker is disposed distal to the reinforcement member.

17. The medical device of claim 15, wherein the second marker is disposed distal to the reinforcement member.

18. A medical device, comprising:
an elongate member having a proximal end, a distal end, and a circumferential wall having an outer surface and an inner surface that defines a lumen;
a reinforcement member disposed within the circumferential wall, extending along a length of the elongate member, and formed of a first material having a first magnetic susceptibility; and
a marker disposed within the circumferential wall distal to the reinforcement member and formed of a second material different from the first material and having a second magnetic susceptibility that is greater than the first magnetic susceptibility.

19. The medical device of claim 18, wherein the second material comprises one of 304V stainless steel and 316LVM stainless steel.

20. A medical device, comprising:
an elongate member having a proximal end, a distal end, and a circumferential wall having an outer surface and an inner surface that defines a lumen;
a reinforcement member comprising a coil disposed within the circumferential wall, extending along a length of the elongate member, and formed of a first material having a first magnetic susceptibility; and
a marker disposed within the circumferential wall distal to the reinforcement member and formed of a second material different from the first material and having a second magnetic susceptibility that is greater than the first magnetic susceptibility.
